(19) United States
(12) Patent Application Publication
(10) Pub. No.: US 2011/0077216 A1
(43) Pub. Date: Mar. 31, 2011

(54) COMPOSITIONS AND METHODS FOR THE TREATMENT OF ATHEROSCLEROSIS AND OTHER RELATED DISEASES

(75) Inventors: Christian Kastrup, Waltham, MA (US); Daniel Griffith Anderson, Sudbury, MA (US); Robert S. Langer, Newton, MA (US); Haeshin Lee, Daejeon (KR); Matthias Nahrendorf, Charlestown, MA (US); Ralph Weissleder, W. Peabody, MA (US); Jose Luiz Figueiredo, Brookline, MA (US)

(73) Assignees: Massachusetts Institute of Technology, Cambridge, MA (US); The General Hospital Corporation d/b/a Massachusetts General Hospital, Boston, MA (US)

(21) Appl. No.: 12/895,278

(22) Filed: Sep. 30, 2010

Related U.S. Application Data
(60) Provisional application No. 61/247,423, filed on Sep. 30, 2009.

(51) Int. Cl. A61K 31/728; A61P 9/10; C08B 37/04; A61K 31/734; C07C 237/20; A61K 31/165

(52) U.S. Cl. 514/54; 536/123.1; 536/18.7; 564/153; 514/616

(57) ABSTRACT

The present invention provides compositions and methods for the treatment of atherosclerosis and other related diseases. In some embodiments, a method comprises providing a composition and forming a coating of the composition on at least a portion of the interior and/or exterior surface of a tissue lumen or other body surface. The composition may remain associated with the tissue lumen or other body surface even in the presence of a strong flow of a fluid (e.g., blood flow in a blood vessel). The composition may associate with the tissue lumen via a plurality of covalent bonds. In some cases, the compositions may comprise at least one additive, for example, a therapeutically active agent or an imaging agent.

### FIELD OF THE INVENTION

The present invention provides compositions of matter which can be used in the treatment of atherosclerosis and other related diseases, and related methods. In some embodiments, the method may comprise providing a composition and forming a coating of the composition on at least a portion the interior and/or exterior surface of a tissue lumen or other body surface. The composition may remain associated with the tissue lumen or other body surface even in the presence of a strong flow of a fluid through the lumen or past the surface (e.g., blood flow in a blood vessel). A composition may associate with a tissue lumen via a plurality of covalent bonds. In some cases, a composition may comprise at least one additive, for example, a therapeutically active agent or an imaging agent.

### BACKGROUND OF THE INVENTION

The hollow or tubular geometry of tissue lumens or hollow organs generally has functional significance such as in the facilitation of fluid or gas transport (blood, urine, lymph, oxygen, or respiratory gases) or cellular containment (ova, sperm). Disease processes may affect a tissue lumen or hollow organ by encroaching upon, obstructing, or otherwise reducing the cross-sectional area of the hollow or tubular elements. Additionally, other disease processes may violate the native boundaries of the hollow organ and thereby affect its barrier function and/or containment ability. Such problems may reduce or severely compromise the ability of the lumen or organ to function properly. Although methods, compositions, and/or systems are known for in vivo paving and/or sealing of an interior surface of a tissue lumen or hollow organ, improved methods and compositions are needed.

### SUMMARY OF THE INVENTION

The present invention is generally related to compositions of matter which can be used in the treatment of atherosclerosis and other related diseases, and related methods. The subject matter of the present invention involves, in some cases, interrelated products, alternative solutions to a particular problem, and/or a plurality of different uses of one or more systems and/or articles.

In one aspect, the invention is directed towards a composition. According to a first set of embodiments, a composition comprises a polysaccharide comprising a plurality of functional groups having the formula:

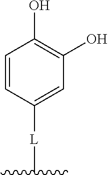

wherein L is a linker associating the functional group to the polysaccharide backbone.

In another aspect, the invention is directed towards a method. According to a first set of embodiments, a method comprises providing a composition comprising a polymer and a plurality of functional groups having the formula:

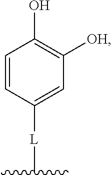

wherein L is a linker associating the functional groups to the polymer, and coating at least a portion of an interior surface of a hollow organ or tissue lumen with the composition.

According to another set of embodiments, a method comprises providing a composition comprising a polymer and a plurality of functional groups having the formula:

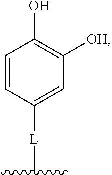

wherein L is a linker associating the functional group to the polymer, and coating at least a portion of a blood vessel plaque and/or aneurism with the composition.

Accordingly to yet another set of embodiments, a method comprises providing a composition comprising a polymer and a plurality of functional groups having the formula:

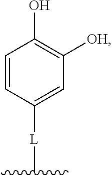

wherein L is a linker associating the functional groups to the polymer, and coating at least a portion of a body surface with the composition.

In some embodiments, a method comprises coating at least a portion of an interior and/or exterior surface of a hollow organ or tissue lumen with a composition covalently attached to the surface.

In other embodiments, a method comprises exposing at least a portion of an interior surface of a hollow organ or tissue lumen to a fluid composition, allowing the fluid composition to at least partially solidify to form a coating covalently attached to the interior surface, and exposing the surface to moving fluid that creates an average shear rate of about 1000 s⁻¹ against the coating, for a period of time of at least about 12 hours, wherein at least about 80% of the coating remains positioned on the surface after about 12 hours.

### BRIEF DESCRIPTION OF THE DRAWINGS

FIG. 1 shows the structure of a composition of the present invention, according to a non-limiting embodiment.

FIG. 2A-2C show non-limiting examples of methods to deliver a composition of the present invention to a tissue lumen.

FIG. 3 shows images of carotid arteries of mice comprising a coating of a composition of the present invention, according to some embodiments.

FIG. 4 is a graph of the release of a therapeutically active agent from a composition of the present invention versus time, according to a non-limiting embodiment.

FIG. 5 shows the structure of a composition of the present invention, according to another non-limiting embodiment.

FIGS. 6A and 6B show timelapse fluorescent images with increasing shear rate of unfunctionalized alginate and functionalized alginate comprising dopamine moieties, respectively, on HUVEC cells.

FIG. 6C shows a graph quantifying the adhesion of a composition of the present invention to glass slides in lap-shear by tension loading, according to a non-limiting embodiment.

FIG. 7A shows an intravital microscopy image of the carotid artery of a live mouse 10 days after coating with an alginate-dopamine composition.

FIG. 7B shows H & E histological sections (top) and fluorescence images (bottom) of a carotid artery coated with an alginate-dopamine composition.

FIG. 7C shows an FMT-CT image of an alginate-dopamine composition inside a carotid artery of a mouse

FIG. 7D shows histological sections (length-wise section) showing a coating of an alginate-dopamine composition along a vessel wall.

FIG. 7E shows intravital microscopy of an alginate-dopamine composition coated on an atherosclerotic plaque.

FIG. 8A shows fluorescence images of a carotid artery in a living mouse painted with an alginate-dopamine composition.

FIG. 8B shows fluorescence images of an alginate-dopamine composition painted in a carotid artery of a mouse at day 1 and day 5 after deposition.

FIG. 8C shows a chart quantifying the amount of an alginate-dopamine composition in a vessel over time.

FIG. 8D shows histological sections of a carotid artery painted with an alginate-dopamine composition for 28 days.

FIG. 8E shows fluorescence images from histological section adjacent to those of FIG. 8D.

FIGS. 9A-9E show fluorescence images of histological section of an alginate-dopamine composition containing fluorescent nanoparticles.

FIGS. 10A-10D show images of painted bifurcation regions of blood vessels using an alginate-dopamine composition comprising degradable particles.

FIG. 11 shows overlaid fluorescence images of histological sections of an alginate-dopamine composition painted on an atherosclerotic plaque in the carotid artery of a mouse.

FIG. 12 shows an image of a carotid artery painted with hyaluronic acid-dopamine.

FIG. 13 shows fluorescence images of a composition of the present invention which was administered to carotid arteries of wild type mice.

FIG. 14 shows fluorescence images of a carotid artery in a living mouse treated with, in some embodiments, a composition of the present invention.

FIG. 15 is a graph of the plague size of treated and untreated mice.

FIGS. 16A-16B show a carotid artery of a mouse coated with alginate-dopamine composition comprising a steroid.

FIGS. 16C-16D show control sample of a carotid artery that was not coated with adhesive hydrogel or steroid.

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

The present invention provides compositions of matter that can have a variety of uses, including application to biological surfaces, and related techniques. Compositions of the invention are applicable to surfaces and can provide better adhesion to surfaces in some instances. In one set of embodiments, compositions can be used for the treatment of atherosclerosis and other related disease, for example, post-angioplasty restenosis, coronary artery diseases, angina, blood vessel plaque, aneurism, and/or other cardiovascular disease in a subject. In some embodiments, a method may comprise providing a composition and forming a coating of the composition on at least a portion of the interior and/or exterior surface of a tissue lumen or other body surface. In some embodiments, a composition has characteristics that allows for the composition to remain associated with the interior and/or exterior surface of a tissue lumen or other body surface, even in the presence of a strong flow of fluid (e.g., blood in a blood vessel). In some cases, a composition may associate with a tissue lumen or other body surface via a plurality of covalent bonds.

In some embodiments, a composition may comprise a polymer and a plurality of functional groups associated with the polymer. The functional groups may aid in the association of the composition with the interior and/or exterior surface of a tissue lumen (or other body surface), for example, by the formation of a plurality of bonds (e.g., covalent bonds). A composition may comprise at least one additive, for example, a therapeutically active agent or an imaging agent. In some cases, the composition may be provided as a fluid composition that may solidify (e.g., to form a solid, a gel, and/or a hydrogel), at least in part, upon exposure to an external stimulus (e.g., a crosslinking agent). In some embodiments, a method may comprise coating at least a portion of an interior and/or exterior surface of a hollow organ or tissue lumen with a composition covalently attached to the surface. In some embodiments, the composition is a hydrogel.

The methods described herein may be applied to a variety of applications. For example, a coating of a composition, as described herein, on the surface of a blood vessel plaque may aid in the healing of plaque by various biochemical mechanisms, including forming a physical barrier between molecules of the blood stream and those of the vasculature. Generally, stents are not used for the treatment of blood vessel plaques that do not significantly impair blood flow and/or stents are not usually suited to render blood vessel plaques less vulnerable and/or inflamed.

The compositions described herein, in some embodiments, are strongly adhesive. The strong adhesive properties are advantageous as the compositions may remain adhered to a tissue lumen (or other body surface) for a longer period of time as compared to a composition which does not have as strong of adhesive properties, especially under a flow of a fluid in the tissue lumen (e.g., blood in a blood vessel). Compositions which are strongly adhered to a tissue lumen (or other body surface) are less likely to become dissociated from the tissue lumen and cause adverse effects. For example, in some instances, a dissociated composition may block downstream blood vessels, embolize, and/or cause tissue ischemia.

Compositions such as those described herein might have been expected, by those of ordinary skill in the art, not to form a coating on the interior and/or exterior surface of a tissue lumen or other body surface as described herein. In contrast, those of ordinary skill in the art would have likely expected the compositions to at least partially block or clog the tissue lumen and/or cause the walls of the tissue lumen to stick together due to the strongly adhesive nature of the compositions. The invention, in part, involves the surprising discovery that the compositions described herein are capable of forming a coating associated with the interior and/or exterior surface of a tissue lumen or other body surface. In addition, during a surgical procedure, the outside of blood vessels may have many forces applied and it would be expected that the forces would cause most materials to detach from the vessel. For example, blood and fluids may wash over the tissue during a surgical procedure, and during closure of the incision, tissues may rub against the vessel. In some embodiments, the compositions of the present invention remain strongly adhere to the vessel during the surgery and/or in the following days when the tissue biologically responds to the composition.

Other beneficial characteristics of the composition and/or methods provided herein may include, but are not limited to: i) promotion of the growth of particular cell types (e.g., endothelial cells), ii) reduction or prevention of excessive growth of cells that would lead to diseases (e.g., such as restenosis), iii) reduction or prevention of immune and/or inflammatory responses, iv) controlled degree of immune and inflammatory responses (e.g., to achieve appropriate healing of a blood vessel plaque, such as by forming healthy scar tissue), v) transportation and/or diffusion of an agent (e.g., a therapeutically active agent) through the composition and/or to a tissue lumen or other body surface, vi) improved mechanical strength of the tissue lumen or other body surface (e.g., so as to match the mechanical strength of surrounding tissue, to provide increased mechanical strength at a blood vessel plaque site), and/or vii) controlled degradation.

Methods may comprise, in some embodiments, forming a coating of a composition on at least a portion of a body surface. For example, in some cases, the body surface is at least a portion of a tissue lumen or hollow organ. The interior and/or exterior of a tissue lumen or hollow organ may be coated with the composition. In some cases, at least a portion of a blood vessel plaque and/or aneurism may be coated with a composition. The interior and/or exterior of the blood vessel plaque and/or aneurism may be coated with the composition.

In some instances, a composition may form a sealing in intimate and conforming contact with, or pave the interior and/or exterior surface of the tissue lumen, hollow organ, blood vessel plaque, and/or aneurism. As used herein, the term “sealing” or “seal” means a coating of sufficiently low porosity that the coating serves a barrier function. The term “paving” refers to coatings which are porous or perforated. The term “tissue lumen” refers any open area inside a body, whether existing naturally, for example, vascular, urological, biliary, esophageal, reproductive, endobronchial, gastrointestinal, and prostatic lumens. Specific non-limiting examples of tissue lumens include arteries (e.g., coronary, femeroiliac, carotid, and vertebrobasilar arteries), veins, ureters, urethrae, bronchi, biliary and pancreatic duct systems, intestines, spermatic tubes, and fallopian tubes. The term “hollow organ” refers to any organ which possesses true spaces including cavities, cavernous sinuses, etc. Non-limiting examples of hollow organs include the heart, liver, kidney, and pancreas. The composition and methods of the present invention may be used with any suitable tissue lumen or hollow organ.

Those of ordinary skill in the art will be aware of methods for coating tissue lumens (or other body surfaces) with a composition, for example, methods for coating a blood vessel with a hydrogel. For example, in some embodiments, a method may comprise providing a fluidic composition, wherein at least a portion of the fluidic composition solidifies to form a coating of the composition on the interior and/or exterior surface of a tissue lumen. In some cases, the fluidic composition may be provided through a catheter (e.g., inside a tissue lumen). The catheter may be a long thin tube which may be manipulated using fluoroscopic guidance, or other means, and may allow for access into the interior of a tissue lumen or hollow organ, and/or to the exterior surface of a body tissue (e.g., tissue lumen, hollow organ, or other tissue). In some cases, the composition may be introduced to the tissue lumen through a simple aperture in the side of the tube, through a raised aperture, or through a shaped nozzle which is extendable away from the surface of the tubular body. Catheters bodies may be made of any suitable material, including, but not limited to, metals (e.g. steel) and thermoplastic polymers. In other cases, the composition may be formed as a coating on at least a portion of a tissue lumen or other body surface using non-catheter techniques, for example, by painting, pipetting, and/or spraying the composition on the tissue lumen or other surface (e.g., interior and/or exterior surface). The coating may be a gel, a hydrogel, and/or a solid. In a particular embodiment, the coating is a hydrogel.

In some embodiments, the method for coating of at least a portion of an interior and/or exterior surface of a tissue lumen (or other body surface) may comprise providing a fluidic composition. In some cases, the fluidic composition may solidify, at least in part, upon exposure to an external stimulus (e.g., a change in temperature, a change in pH, a cross-linking agent). In some cases, the composition may partially solidify to form a gel, a hydrogel, and/or a solid. As used herein, the term “gel” is given its ordinary meaning in the art and refers to a composition comprising a polymer network that is able to trap and contain fluids. The term “hydrogel” is given its ordinary meaning in the art and refers to a class of polymeric compositions which are extensively swollen in an aqueous medium but which do not dissolve in them. It should be understood, however, that a composition in a gel-like state may comprise isolated areas wherein the composition exists in a solid state. Methods for solidifying a composition, at least in part, are described herein. In some embodiments, the external stimulus is not light and/or a photoinitiator.

In embodiments where the composition is provided using a catheter, a preconditioning solution may be delivered through the catheter prior to delivery of the composition (e.g., fluid composition). In some cases, the preconditioning solution may facilitate the formation of the composition on the interior surface of the tissue lumen. For example, the fluid composition may have a higher surface tension with the preconditioning solution than the tissue lumen, thereby causing the composition to preferentially wet the interior surface of the tissue lumen. Those of ordinary skill in the art will be able to select appropriate preconditioning solutions based upon the properties of the composition (e.g., based upon surface tension values). A non-limiting example of a preconditioning solution includes perfluorocarbon oil. In some embodiments, the preconditioning solution may be non-toxic or substantially non-toxic, bioadsorbable, and/or biodegradable.

In some cases, the composition may associate with the tissue lumen by the formation of at least one bond, such as an ionic bond, a covalent bond (e.g., carbon-carbon, carbon-oxygen, oxygen-silicon, sulfur-sulfur, phosphorus-nitrogen, carbon-nitrogen, metal-oxygen or other covalent bonds), a hydrogen bond (e.g., between hydroxyl, amine, carboxyl, thiol and/or similar functional groups, for example), a dative bond (e.g., complexation or chelation between metal ions and monodentate or multidentate ligands), Van der Waals interactions, or the like. In a particular example, the composition may associate with the tissue lumen by the formation of a plurality of covalent bonds. The term “covalent bond” is given its ordinary meaning in the art and refers to the type of bonding in which the electronegativity difference between the bonded atoms is small or non-existent, resulting in the sharing of an electron between two atoms. For example, non-limiting examples of covalent bonds include carbon-carbon bonds, carbon-oxygen bonds, and carbon-hydrogen bonds. A covalent bond be multiple or single bond.

In some cases, following association of the composition with the interior and/or exterior surface of a tissue lumen (or other body surface), the composition may be exposed to a moving fluid (e.g., blood in a blood vessel). The composition may be strongly associated (e.g., adhered) with the tissue lumen such that the composition may not substantially dissociate. For example, the moving fluid may create an average shear rate of about 10 s⁻¹, about 25 s⁻¹, about 50 s⁻¹, about 100 s⁻¹, about 200 s⁻¹, about 300 s⁻¹, about 400 s⁻¹, 500 s⁻¹, about 1000 s⁻¹, about 2000 s⁻¹, about 3000 s⁻¹, about 4000 s⁻¹, about 5000 s⁻¹, about 7500 s⁻¹, about 10,000 s⁻¹, about 15,000 s⁻¹, about 17,000 s⁻¹, about 20,000 s⁻¹⁻, or greater, against the coating, for a period of time of at least about 1 minutes, about 5 minutes, about 10 minutes, about 30 minutes, 1 hours, about 2 hours, about 4 hours, about 8 hours, about 24 hours, about 48 hours, about 7 days, about 14 days, about 1 month, or longer, wherein at least about 60%, about 70%, about 75%, about 80%, about 85%, about 90%, about 95%, about 98%, or more, of the composition remains positioned on the surface after the period of time. In a particular embodiment, the moving fluid may create an average shear rate of about 1000 s⁻¹ against the coating, for a period of time of at least about 12 hours, wherein at least about 80% of the coating remains positioned on the surface after about 12 hours. In some embodiments, the strong association (e.g., adhesion) of the composition to the interior and/or exterior surface of the tissue lumen (or other body surface) may be, at least in part, attributed to a plurality of covalent bonds between the composition and the tissue lumen. The term “average shear rate” is given its ordinary meaning in the art and refers to the average ratio between velocity of a liquid and the distance between the two shearing planes (e.g., blood vessel walls).

The thickness of the coating of the composition associated with the interior and/or exterior surface of a tissue lumen or other body surface may be varied using skills known to those of ordinary skill in the art. For example, the amount of composition provided may be increased or decreased to modify the thickness of the coating. The thickness of the coating may or may not be substantially uniform. In some cases, the thickness of the coating may be about 0.1 nm, about 0.5 nm, about 1 nm, about 5 nm, about 10 nm, about 100 nm, about 1 um (micrometer), about 2 um, about 5 um, about 25 um, about 50 um, about 100 um, about 250 um, about 500 um, about 750 um, about 1 mm, about 2 mm, or the like, or any range therein (e.g., between about 1 nm and about 1 mm, between about 1 um and about 1 mm, between about 1 um and about 1 um, between about 100 nm and about 100 um, or the like). The thickness of the coating may be varied based upon the desired application of the coating. For example, thinner coatings may be better suited to function as a sealant and/or partitioning barrier, bandage, and/or drug depot whereas thicker coatings may provide increased structural support.

In some cases, the composition comprises a polymer. The polymer may form a fluid composition and the fluid composition may solidify, at least in part, upon exposure to an external stimulus. In some cases, the external stimulus may be a change in the pH of the surrounding environment. For example, the pH of the surrounding environment may be increased or decreased, thereby stimulating the polymer to crosslink. In other instances, the external stimulus may be a crosslinking agent. Upon exposure to the crosslinking agent, crosslinks may form between the polymer chains, thereby solidify, at least in part, the fluid composition. The polymer chains may be crosslinked using methods and techniques known to those of ordinary skill in the art. In a non-limiting embodiment, prior to (e.g., immediately prior to, or about 0.1 sec, about 1 sec, about 2 sec, about 5 sec, about 10 sec, about 30 sec, about 1 min, about 2 min, about 5 min, about 10 min, etc., prior to) the introduction of a fluid composition comprising the polymer into a tissue lumen or other body surface, the fluid composition may be exposed to a crosslinking agent. That is, the fluid composition may be mixed with or simultaneously provided to the tissue lumen with a crosslinking agent (e.g., a solution comprising the crosslinking agent) or another external stimulus (e.g., a solution which affects the pH of the environment surrounding the polymer). The term “crosslinking agent” is defined herein to include any reagent, molecule, atom, or ion that is capable of forming one or more crosslinks between molecules of a polymer and/or between one or more atoms in a single molecule of a polymer, or alternatively, a reagent, molecule, atom, or ion which is capable of promoting the activation of at least one section of a polymer chain such that the at least one section may react with a section (e.g., activated or non-activated section) of a different polymer chain, thereby forming a crosslink. For example, in embodiments where the polymer comprising dopamine moieties, the crosslinking agent may oxidize at least a portion of the dopamine moieties, and an oxidized dopamine molecule may react with another oxidized dopamine molecule from a different polymeric chain, thereby forming a crosslink between the two polymer chains (e.g., by forming a bond between a dopamine molecule on one polymer chain and a dopamine molecule on the other polymer chain).

The rate or degree of crosslinking of a polymer may depend on numerous factors including, but not limited to, the amount and nature of the crosslinking agent provided, the temperature, and the pH of the surrounding environment. These and other factors may be varied to increase or decrease the time a composition (e.g., comprising a polymer) takes to solidify, at least in part. In some cases, the composition may solidify, at least in part, in about 1 minute, about 2 minutes, about 3 minutes, about 5 minutes, about 10 minutes, about 15 minutes, about 20 minutes, about 30 minutes, about 1 hour, or more, following exposure to an external stimulus. In addition, the degree of crosslinking may be varied to tailor the properties of the solidified composition. For example, a highly crosslinked composition generally may be structurally stronger as compared with a lightly crosslinked composition. In the design of compositions for a particular application, the degree of crosslinking may be adjusted to achieve the desired compromise between, for example, rate of solidifying and/or level of structural integrity. In some cases, the amount of crosslinking of the polymer may be adjusted to allow for a certain controlled rate of release of additives (e.g., therapeutically active agent, cells, imaging agents) from the composition. Those of ordinary skill in the art will be aware of techniques and methods for modulating the degree of crosslinking in such materials and selecting the appropriate crosslinking agent based upon the polymer composition.

In some embodiments, the composition (e.g., fluid composition) may be provided as a solution. Those of ordinary skill in the art will be aware of appropriate solvents the composition may be dispersed or dissolved in, for example, solvents which are non-toxic or substantially non-toxic. Non-limiting examples of solvents include water, alcohols, or combinations thereof. In some cases, a solution comprising the composition may comprise at least one additive, as described herein, wherein at least a portion of the additive is contained by the composition upon the solidification of the composition in the tissue lumen or other body surface. For example, at least about 30%, about 40%, about 50%, about 60%, about 70%, about 80%, about 90%, about 95%, about 99%, or more, of the additive provided is contained in the composition associated with the tissue lumen or other body surface (e.g., upon solidification of the composition).

In some embodiments, a composition comprises a plurality of functional groups and a polymer, wherein the plurality of functional groups is associated with the polymer. The functional groups may aid in the association of the composition with the interior and/or exterior surface of the tissue lumen or other body surface, for example, by the formation of at least one bond (e.g., covalent bond), as described herein. The term “functional group” is given its ordinary meaning in the art and refers to groups of atoms that give a composition, compound, or substance to which they are linked characteristic chemical and physical properties. For example, in connection with the present invention, in some cases, a functional group may be identifiable sites, regions, or functional portions of the composition that contributes to the associative process of the composition with a tissue lumen or other body surface. The composition may comprise between about 2 and about 10,000, or between about 2 and about 5,000, or between about 2 and 1,000, or between 2 and about 5,00, or between 10 and 500, or between 10 and 100, etc., functional groups. In some embodiments, the composition comprises at least 2, or at least 3, or at least 4, or at least 5, or at least 10, or at least 20, or at least 30, or at least 50, or at least 100, or at least 500, or more, functional groups.

In some embodiments, a functional group associated with the polymer may comprise a dopamine (e.g., 3,4-dihydroxyphenethylamine) or a 3,4-dihydroxyphenylalanine group. A dopamine or 3,4-dihydroxyphenylalanine moiety may form a covalent bond with a tissue lumen or other body surface. As a non-limiting example of a possible reaction that may occur, the catechol group of a dopamine or 3,4-dihydroxyphenylalamine moiety may be oxidized to form a reactive quinone, which may reaction with nucleophiles (e.g., amines, hydroxyl groups, thiols) comprised in the lumen (e.g., via a Michael Addition reaction).

In some embodiments, a composition may comprise a polymer (e.g., a polysaccharide) comprising a plurality of functional groups having the formula:

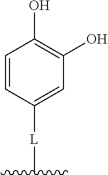

wherein L is a linker associating the functional group to the polymer (e.g., polysaccharide) backbone. The linker may be any molecule that may form a linkage between the functional group and the polymer backbone. For example, the linker may be an alkyl group, a heteroalkyl group, an aryl group, a heteroaryl group, an acyl group, or the like, optionally substituted. In some cases, the functional groups may have the formula:

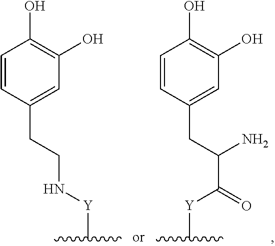

wherein Y may be a linker linking the functional group to the polymer (e.g., polysaccharide) backbone, or optionally absent.

A “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer is biologically derived, i.e., a biopolymer. In some cases, additional moieties may also be present in the polymer, for example functional groups such as those described herein.

If more than one type of repeat unit is present within the polymer, then the polymer is said to be a “copolymer.” It is to be understood that in any embodiment employing a polymer, the polymer being employed may be a copolymer. The repeat units forming the copolymer may be arranged in any fashion. For example, the repeat units may be arranged in a random order, in an alternating order, or as a “block” copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In some embodiments, the polymer may be a polysaccharide. Non-limiting examples of polysaccharides include starches, starch derivatives, modified starches, cellulose derivatives, naturally occurring gums, and derivatives thereof, biopolymers, and the like. Exemplary starch derivatives and modified starches include pregelatinized starches, crosslinked starches, dextrinized starches, oxidized starches, degraded starches, such as maltodextrins, starch ethers such as carboxymethyl starch, hydroxyethyl starch, hydroxypropyl starch, cationic starches, and the like, and starch esters such as starch acetate. Exemplary cellulose ethers include carboxymethyl cellulose, hydroxyethyl cellulose, hydroxypropyl cellulose, carboxymethyl hydroxyethyl cellulose, methyl cellulose, cationic celluloses, and the like. Exemplary gums and derivatives thereof include guar gum, hydroxypropyl guar, carboxymethyl hydroxypropyl guar, locust bean gum, ghatti gum, karaya gum, tamarind gum, carrageenan, alginates, and the like.

Other non-limiting types of polymers may include co-polymers or polymers having carboxylic acid groups such as glycolic acid and lactic acid, polyurethanes, polyesters such as poly(ethylene terephthalate), polyamides such as nylon, polyacrylonitriles, polyphosphazines, polylactones such as polycaprolactone, and polyanhydrides such as poly[bis(p-carboxyphenoxy)propane anhydride] and other polymers or copolymers such as polyethylene, polyvinyl chloride and ethylene vinyl acetate. In some cases, the polymer may be hyaluronic acid or polyethylene glycol.

In one set of embodiments, the polymer (or composition) may be biocompatible, i.e., the polymer that does not typically induce an adverse response when inserted or injected into a living subject, for example, without significant inflammation and/or acute rejection of the polymer by the immune system, for instance, via a T-cell response. It will be recognized, of course, that “biocompatibility” is a relative term, and some degree of immune response is to be expected even for polymers that are highly compatible with living tissue. However, as used herein, “biocompatibility” refers to the acute rejection of composition by at least a portion of the immune system, i.e., a non-biocompatible composition implanted into a subject provokes an immune response in the subject that is severe enough such that the rejection of the composition by the immune system cannot be adequately controlled, and often is of a degree such that the composition must be removed from the subject. One simple test to determine biocompatibility is to expose a polymer to cells in vitro; biocompatible polymers are polymers that typically will not result in significant cell death at moderate concentrations, e.g., at concentrations of about 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments of the present invention include polydioxanone (PDO), polyhydroxyalkanoate, polyhydroxybutyrate, poly(glycerol sebacate), polyglycolide, polylactide, polycaprolactone, or copolymers or derivatives including these and/or other polymers.

In certain embodiments, the biocompatible polymer (or composition) may be biodegradable, i.e., the polymer is able to degrade, chemically and/or biologically, within a physiological environment, such as within the body. For instance, the polymer may be one that hydrolyzes spontaneously upon exposure to water (e.g., within a subject), the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer is degraded into monomers and/or other nonpolymeric moieties) may be on the order of hours, days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.). Examples of biodegradable polymers include, but are not limited to, poly(lactide) (or poly(lactic acid)), poly(glycolide) (or poly(glycolic acid)), poly(orthoesters), poly(caprolactones), polylysine, poly(ethylene imine), poly(acrylic acid), poly(urethanes), poly(anhydrides), poly(esters), poly(trimethylene carbonate), poly(ethyleneimine), poly(acrylic acid), poly(urethane), poly(beta amino esters) or the like, and copolymers or derivatives of these and/or other polymers, for example, poly(lactide-co-glycolide) (PLGA).

Those of ordinary skill in the art will be aware of methods for synthesizing a composition comprising a polymer and a plurality of functional groups associated with the polymer backbone. In some cases, the polymer may comprise a plurality of functional groups upon formation of the polymer (e.g., the monomers which are polymerized comprise the functional groups). In other cases, a polymer may be functionalized with the plurality of functional groups using chemical reagents. For example, a polymer may comprise monomeric units comprising reactive groups (e.g., carboxylic acid groups, hydroxyl groups) which may be reacted with a functional group precursor under suitable conditions (e.g., temperature, catalysts, acidic or basic conditions, etc.), such that functional groups become associated with at least a portion of the reactive groups of the monomer units. As a specific example, a monomer unit may comprise a —COOH group and the functional group may comprise an —NH₃Cl group, wherein the two groups may react to form a bond between the monomeric unit and the functional group (e.g., —COO—NH₂—) and a reaction byproduct (e.g., HCl). As another specific example, the polymer may comprise an alginate backbone, and the functional group precursor may be 3-hydroxytyramine hydrochloride. Under basic conditions, a functionalized polymer may form having the structure:

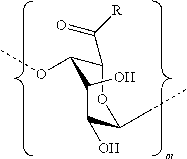

wherein m is an integer between 1 and 10,000 and each R is OH or a group having the structure:

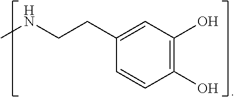

In some cases, m is an integer between about 1 and about 5,000, between about 100 and about 10,000, between about 100 and about 5,000, between about 500 and about 5000, between about 1000 and about 5000, or any range therein. Compositions comprising an alginate backbone and dopamine functions groups may be beneficial as i) unmodified alginate is biocompatible and used clinically, ii) solidification may be controlled various mechanisms including crosslinking via the dopamine moieties and/or via ionic interactions using divalent cations, and iii) a relatively functional group substitution may be achieved.

Those of ordinary skill in the art will be able to determine appropriate ratios of reagents (e.g., ratio of reactive groups per monomeric units in the polymer to functional group precursor) such that the desired number of reactive groups in the polymer become functionalized with a functional group. In some cases, the ratio of functionalized to unfunctionalized reactive groups per monomeric unit in the polymer is about 1:1, about 1:2, about 1:3, about 1:4, about 1:5, about 1:8, about 1:10, about 1:20, about 1:50, or more. The ratio of functionalized to unfunctionalized reactive groups in the polymer may affect the strength of the association of the polymer with a tissue lumen or other body surface. For example, a polymer comprising a higher number of functional groups (e.g., which are able to associate by formation of a bond with a tissue lumen) may be more strongly associated with the tissue lumen as compared to a polymer comprising a lower number of functional groups.

As another non-limiting example, the functionalized polymer may have the structure:

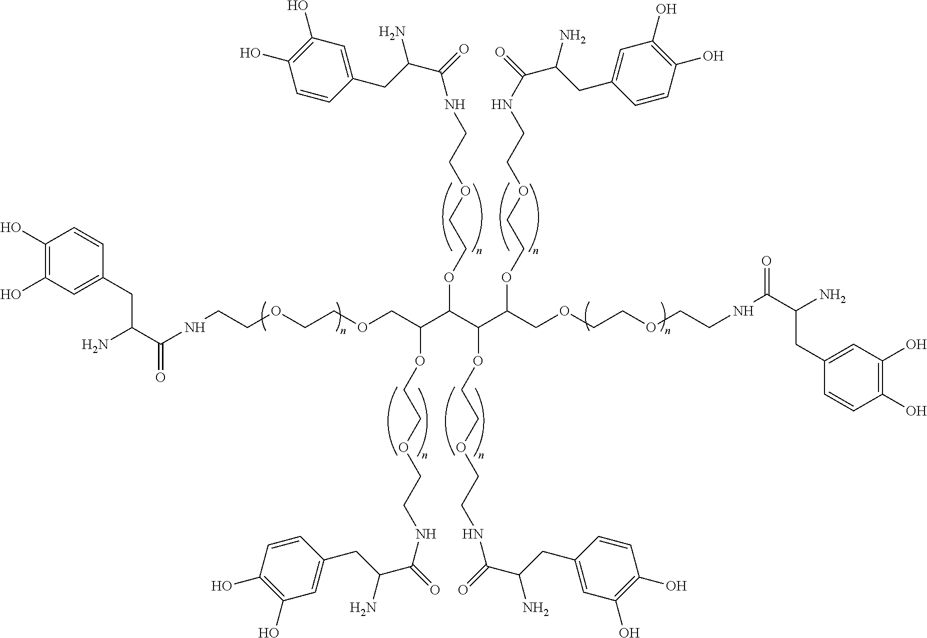

wherein each n can be the same or different and is an integer between 1 and about 100. In some cases, n may be between about 1 and about 50, between about 10 and about 100, between about 25 and about 100, between about 50 and about 100, between about 25 and about 75, between about 1 and about 500, between about 1 and about 250, between about 10 and about 500, between about 10 and about 250, between about 50 and about 250, or the like. That is, in some embodiments, the polymer may comprise a polyethylene glycol backbone and the functional groups may comprise 3,4-dihydroxyphenylalanine moieties. In this particular structure, the molecule comprises six polyethylene glycol chains radiating from a central group. Those of ordinary skill in the art will be able to recognize other possible compositions comprising a polyethylene glycol backbone and 3,4-dihydroxyphenylalanine functional groups. For example, the central portion of the molecule may comprise more or less arms (e.g., 2, 3, 4, 5, 7, 8, 9, 10, etc., arms) or more than one central groups (e.g., 2 central groups associated with each other through one arm, and each comprising 2, 3, 4, 5, 6, etc., or more arms), as compared to the structure above.

In some cases, a composition may comprise one or more additives. The additive may be a therapeutically active agent, an imaging agent, and/or a diagnostic agent. In some embodiments, the composition may comprise more than one agent, for example, at least two agents, at least three agents, at least four agents, or more. Non-limiting examples of additives include proteins, peptides, polysaccharides, lipids, nucleic acid molecules, synthetic organic molecules, hormones, chemotherapeutics, antibiotics, antivirals, antifungals, vasoactive compounds, immunomodulatory compounds, vaccines, local anesthetics, antiangiogenic agents, and antibodies. Non-limiting examples of diagnostic agents include gas, radiolabels, magnetic particles, radioopaque compounds, and other materials known to those skilled in the art.

In some cases, upon association of the composition with a tissue lumen or other body surface, an additive (e.g., therapeutically active agent) may be locally delivered to the tissue lumen or body surface the composition is associated with. In some cases, localized delivery of a therapeutically active agent may be advantageous over systemic delivery. For example, in some cases, a higher concentration of the therapeutically active agent may be delivered with less toxic effects. Also, some therapeutically active agents may be delivered more readily if the agent is hydrophobic, degraded, and/or metabolized rapidly, etc. As a non-limiting example, a composition may be associated with a blood vessel plaque and the therapeutically active agent may function by making the plaque less vulnerable. As yet another example, a composition may deliver a therapeutically active agent to diseased vessels such as those that feed tumors or aneurisms, and other lumens such as the intestine for intestinal cancer and inflammatory bowl disease. In some cases, the agent (e.g., therapeutically active agent) may also be released, at least in part, into the fluid flowing in the tissue lumen (e.g., to the blood flowing in a blood vessel). The composition may contain a therapeutically active amount of the therapeutically active agent.

In some cases, the therapeutically active agent may be selected to aid in the treatment of a tissue lumen or other body surface to which the composition is to be associated with (e.g., for treatment of a blood vessel plaque). Non-limiting examples of therapeutically active agents for use in coronary artery applications are anti-thrombotic agents (e.g., prostacyclin and salicylates), thrombolytic agents (e.g., streptokinase, urokinase, tissue plasminogen activator (TPA), anisoylated plasminogen-streptokinase activator complex (APSAC)), vasodilating agents (e.g., nitrates and calcium channel blocking drugs), anti-proliferative agents (e.g., colchicine and alkylating agents), intercalating agents, growth modulating factors (e.g., interleukins), transformation growth factor beta, congeners of platelet derived growth factor and monoclonal antibodies directed against growth factors, anti-thrombotic agents (e.g., anti-GIIb/3a, trigramin, prostacyclin, salicylates), thrombolytic agents (e.g., streptokinase, urokinase, tissue plasminogen activator (TPA), anisoylated plasminogen-streptokinase activator complex (APSAC)), anti-inflammatory agents (e.g., both steroidal and non-steroidal), and other agents which may modulate vessel tone, function, arteriosclerosis, and/or healing response to vessel or organ injury post intervention. Anti-proliferative agent or high efficacy anti-inflammatory agent may be also useful for treatment of focal vasculitides and/or other inflammatory arteritidies (e.g., granulomatous arteritis, polyarteritis nodosa, temporal arteritis, Wegener's granulomatosis). Anti-inflammatory agents may also be useful in connection with indications such as inflammatory bowel disease, Crohn's disease, ulcerative colitis, or focal GI inflammatory diseases. A non-limiting example of a therapeutically active agent is methylprednisilone.

In some cases, the additive may be a plurality of cells. For examples, the cells may be selected, or designed using principles of recombinant DNA technology, to produce specific agents such as growth factors. In such a way, a continuously regenerating supply of a therapeutic agent may be provided without concerns for stability, initial overdosing, and the like. Cells incorporated in a composition may also be progenitor cells corresponding to the type of tissue in the lumen treated or other cells providing therapeutic advantage. For example, liver cells might be incorporated into a composition within a lumen created in the liver of a patient to facilitate regeneration and closure of that lumen. This might be an appropriate therapy in the case where scar tissue or other diseased, e.g. cirrhosis, fibrosis, cystic disease or malignancy, or non-functional tissue segment has formed in the liver or other organ and must be removed.

In some cases, the additive may be an imaging agent which aids in the visualization and/or monitoring of the composition. For example, additive may be a chemical such as barium, iodine, or tantalum salts for X-ray radio-opacity allow visualization and monitoring of the coating. As another example, a plurality of fluorescent particles (e.g., microparticles, nanoparticles) may be incorporated or contained within the composition and the composition may be examined using fluorescent spectroscopy.

In some embodiments, the compositions of the present invention may be used to treat and/or prevent atherosclerosis and other related disease, for example, post-angioplasty restenosis, coronary artery diseases, angina, blood vessel plaque, aneurism, and/or other cardiovascular disease in a subject. In some cases, coating the exterior of a surface with a composition of the present invention may be useful for treating or preventing damaged portions of vessels. For example, the compositions can be used to treat regions where blood vessels are surgically connected together (anastomoses), where vessels are connected to artificial grafts, and/or to treat areas where blood vessels have been punctured. In such embodiments, the compositions may further comprise a therapeutically active agent which can aid in vessel healing.

The invention further comprises preparations, formulations, kits, and the like, comprising any of the compositions as described herein. In some cases, treatment of a disease may involve the use of compositions as described herein. That is, one aspect of the invention involves a series of compositions useful for treatment of a disease. These compositions may also be packaged in kits, optionally including instructions for use of the composition for the treatment of such conditions. These and other embodiments of the invention may also involve promotion of the treatment of a disease according to any of the techniques and compositions and combinations of compositions described herein.

The kit, in one set of embodiments, may comprise a carrier means being compartmentalized to receive in close confinement one or more container means such as vials, tubes, and the like, each of the container means comprising one of the separate elements to be used in the method. For example, one of the container means may comprise a positive control in the assay. Additionally, the kit may include containers for other components, for example, buffers useful in the assay.

The term “atherosclerosis” is given its ordinary meaning in the art and refers to a disease of the arterial wall in which the layer thickens, causing narrowing of the channel and thus, impairing blood flow. Atherosclerosis may occur in any area of the body, but can be most damaging to a subject when it occurs in the heart, brain or blood vessels leading to the brain stem. Atherosclerosis includes thickening and hardening of artery walls or the accumulation of fat, cholesterol and other substances that form atheromas or plaques. Atherosclerosis may result also from calcification, hemorrhage, ulceration, thrombosis, and/or trauma. The term “blood vessel plaque” refers to fatty deposits and/or any other type of build-up that can cause or contribute to stenosis or occlusion within a blood vessel, such as calcium deposits. The term “aneurism” refers to the abnormal enlargement or bulging of an artery caused by damage to or weakness in the blood vessel wall. Although aneurysms can occur in any type of the body's blood vessels, they almost always form in an artery.

As used herein, a “subject” or a “patient” refers to any mammal (e.g., a human), such as a mammal that comprises at least one tissue lumen or hollow organ. Examples include a human, a non-human primate, a cow, a horse, a pig, a sheep, a goat, a dog, a cat, or a rodent such as a mouse, a rat, a hamster, or a guinea pig. Generally, or course, the invention is directed toward use with humans. A subject may be a subject diagnosed with the disease or condition or otherwise known to have the disease or condition (e.g., atherosclerosis). In some embodiments, a subject may be diagnosed as, or known to be, at risk of developing a disease or condition.

The phrase “therapeutically effective amount” as used herein means that amount of a compound, material, or composition comprising a compound of the present invention which is effective for producing some desired therapeutic effect in a subject at a reasonable benefit/risk ratio applicable to any medical treatment. Accordingly, a therapeutically effective amount prevents, minimizes, or reverses disease progression associated with a disease or condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

In the compounds and compositions of the invention, the term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, a straight chain or branched chain alkyl may have 30 or fewer carbon atoms in its backbone, and, in some cases, 20, or fewer. In some embodiments, a straight chain or branched chain alkyl may have 12 or fewer carbon atoms in its backbone (e.g., C₁-C₁₂ for straight chain, C₃-C₁₂ for branched chain), 6 or fewer, or 4 or fewer. Likewise, cycloalkyls may have from 3-10 carbon atoms in their ring structure, or 5, 6, or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The term “heteroalkyl” refers to an alkyl group as described herein in which one or more carbon atoms is replaced by a heteroatom. Suitable heteroatoms include oxygen, sulfur, nitrogen, phosphorus, and the like. Examples of heteroalkyl groups include, but are not limited to, alkoxy, amino, thioester, and the like.

The terms “alkenyl” and “alkynyl” refer to unsaturated aliphatic groups analogous in length and possible substitution to the alkyls described above, but that contain at least one double or triple bond respectively.

The terms “heteroalkenyl” and “heteroalkynyl” refer to unsaturated aliphatic groups analogous in length and possible substitution to the heteroalkyls described above, but that contain at least one double or triple bond respectively.

As used herein, the term “halogen” or “halide” designates —F, —Cl, —Br, or —I.

The terms “carboxyl group,” “carbonyl group,” and “acyl group” are recognized in the art and can include such moieties as can be represented by the general formula:

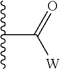

wherein W is H, OH, O-alkyl, O-alkenyl, or a salt thereof. Where W is O-alkyl, the formula represents an “ester.” Where W is OH, the formula represents a “carboxylic acid.” The term “carboxylate” refers to an anionic carboxyl group. In general, where the oxygen atom of the above formula is replaced by sulfur, the formula represents a “thiolcarbonyl” group. Where W is a S-alkyl, the formula represents a “thiolester.” Where W is SH, the formula represents a “thiolcarboxylic acid.” On the other hand, where W is alkyl, the above formula represents a “ketone” group. Where W is hydrogen, the above formula represents an “aldehyde” group.

The term “aryl” refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls, and/or heterocyclyls. The aryl group may be optionally substituted, as described herein. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl groups. In some cases, the

The term “alkoxy” refers to the group, —O-alkyl.

The term “aryloxy” refers to the group, —O-aryl.

The term “acyloxy” refers to the group, —O-acyl.

The term “aralkyl” or “arylalkyl,” as used herein, refers to an alkyl group substituted with an aryl group.

The terms “heteroaryl” refers to aryl groups comprising at least one heteroatom as a ring atom.

The term “heterocycle” refers to refer to cyclic groups containing at least one heteroatom as a ring atom, in some cases, 1 to 3 heteroatoms as ring atoms, with the remainder of the ring atoms being carbon atoms. Suitable heteroatoms include oxygen, sulfur, nitrogen, phosphorus, and the like. In some cases, the heterocycle may be 3- to 10-membered ring structures or 3- to 7-membered rings, whose ring structures include one to four heteroatoms. The term “heterocycle” may include heteroaryl groups, saturated heterocycles (e.g., cycloheteroalkyl) groups, or combinations thereof. The heterocycle may be a saturated molecule, or may comprise one or more double bonds. In some case, the heterocycle is a nitrogen heterocycle, wherein at least one ring comprises at least one nitrogen ring atom. The heterocycles may be fused to other rings to form a polycylic heterocycle. The heterocycle may also be fused to a spirocyclic group. In some cases, the heterocycle may be attached to a compound via a nitrogen or a carbon atom in the ring.

The terms “amine” and “amino” are art-recognized and refer to both unsubstituted and substituted amines, e.g., a moiety that can be represented by the general formula: N(R′)(R″)(R′″) wherein R′, R″, and R′″ each independently represent a group permitted by the rules of valence. An example of a substituted amine is benzylamine.

Any of the above groups may be optionally substituted. As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl group” must still comprise the phenyl moiety and can not be modified by substitution, in this definition, to become, e.g., a pyridine ring. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms.

Examples of substituents include, but are not limited to, halogen, azide, alkyl, aralkyl, alkenyl, alkynyl, cycloalkyl, hydroxyl, alkoxyl, amino, nitro, sulfhydryl, amino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, -carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, -carboxamidoaryl, hydroxyalkyl, haloalkyl, alkylaminoalkylcarboxy-, aminocarboxamidoalkyl-, cyano, alkoxyalkyl, perhaloalkyl, arylalkyloxyalkyl, and the like.

The following examples are intended to illustrate certain embodiments of the present invention, but do not exemplify the full scope of the invention.

### Example 1

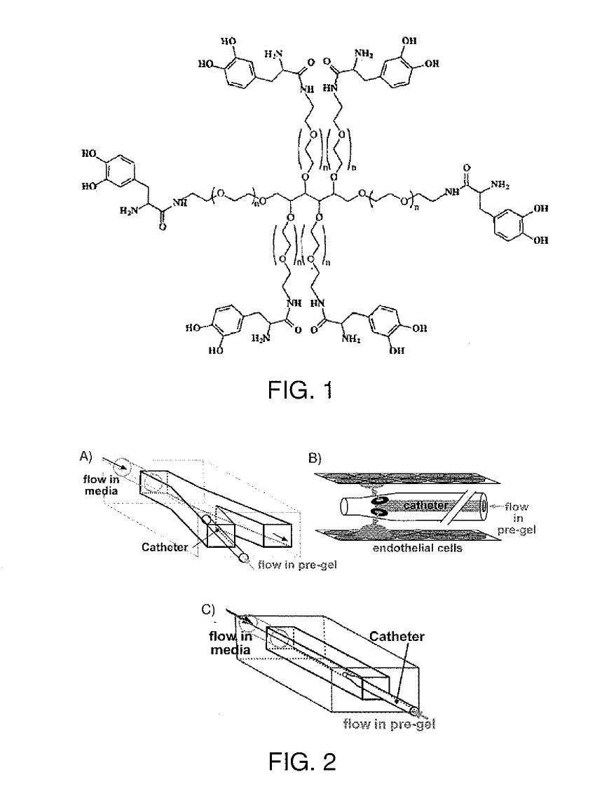

The following examples described compositions and methods comprising PEG-DOPA. The chemical structure of PEG-DOPA monomer of comprises of six 3,4-dihydroxy-L-phenylalanine (DOPA) moieties coupled to a six arm polyethyleneglycol (PEG) molecule (FIG. 1). PEG was used as a backbone for the DOPA moieties to provide biocompatibility and a hydrogel matrix. As described herein, other hydrogels, in addition to PEG, may be used to connect the DOPA moieties, such as alginate derivatives, hyaluronic acid, and other extracellular matrix materials. In addition, peptide sequences, such as RGD can be linked to the monomer to change the properties of the gel. To achieve controllable degradation ester linkages and other degradable linkages may be incorporated into the gel. These variables may be varied to achieve the following properties: i) strong adhesion to vessel wall, ii) allow the growth of particular cell types, such as endothelial cells, iii) prevent excessive growth of cells that would lead to restenosis, iv) prevent strong immune and inflammatory responses, v) control the degree of immune and inflammatory responses to achieve appropriate healing of the plaque area, such as by forming healthy scar tissue, vi) appropriate transport properties to allow specific nutrient and therapeutics to diffuse through the gel, vii) appropriate mechanical strength, such as to match the mechanical strength of surrounding tissue or to provide increased mechanical strength at the plaque site, and viii) controlled degradation.

The composition may be delivered to the vasculature via an intravascular catheter. The monomer of the hydrogel was delivered in a liquid form through a catheter inside of the blood vessel (FIG. 2B). The gel hardened in the blood vessel and coated the surface of the blood vessel. To facilitate a uniform coating of the gel on the vessel, in this embodiment, the vessel was first rinsed with a perfluorocarbon oil. The aqueous hydrogel had a higher surface tension with the perfluorocarbon oil than the vessel wall, and thus the hydrogel preferentially wetted the vessel wall and formed an even coating. This method may also be used to coat the outside of blood vessels with a hydrogel drug depot to deliver drugs to the vascular wall, as described herein.

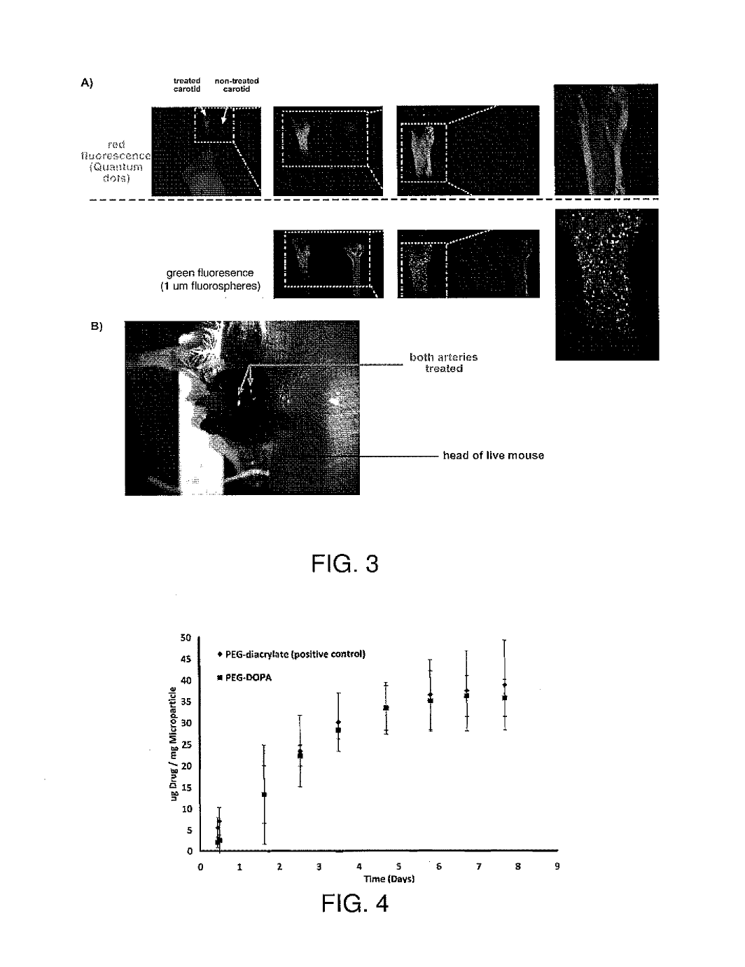

The hydrogel adhered to the carotid arteries of mice. Using the hydrogel and delivery method described in this example, the hydrogel was coated on the carotid arteries of mice (FIG. 3). The gel contained nanoparticles (red) and 1 mm microparticles (green) which were used to image the location of the gel in the arteries (FIG. 3A). This technique was also used to coat both of the carotid arteries of mice (FIG. 3B). After coating both of carotid arteries of mice, the blood flow through the arteries to the brain remained high. Specifically, FIG. 3 shows (A) a treated and untreated carotid artery of a mouse, wherein the gel contained nanoparticles (top images) and 1 mm microparticles (bottom images), and where (B) both carotid arteries of a live mouse treated, demonstrating sufficient flow through the arteries occurred. The arrows indicate the location of the gel (white) in the arteries.

To demonstrate that an adhesive gel may be used as a drug depot, where therapeutic incorporated in the gel may be released, a model drug, methylprednisilone, was encapsulated in degradable particles. This model drug was released from the gel with an identical profile to its release from a non-adhesive PEG-diacrylate gel that was crosslinked using UV light (FIG. 4). Specifically, FIG. 4 shows the release of a methylprednisilone from the adhesive PEG-DOPA hydrogel and a control PEG-diacrylate hydrogel. Methylprednisilone was incorporated into degradable microparticles of poly(lactic-co-glycolic acid) which were blended into the hydrogels.

### Example 2

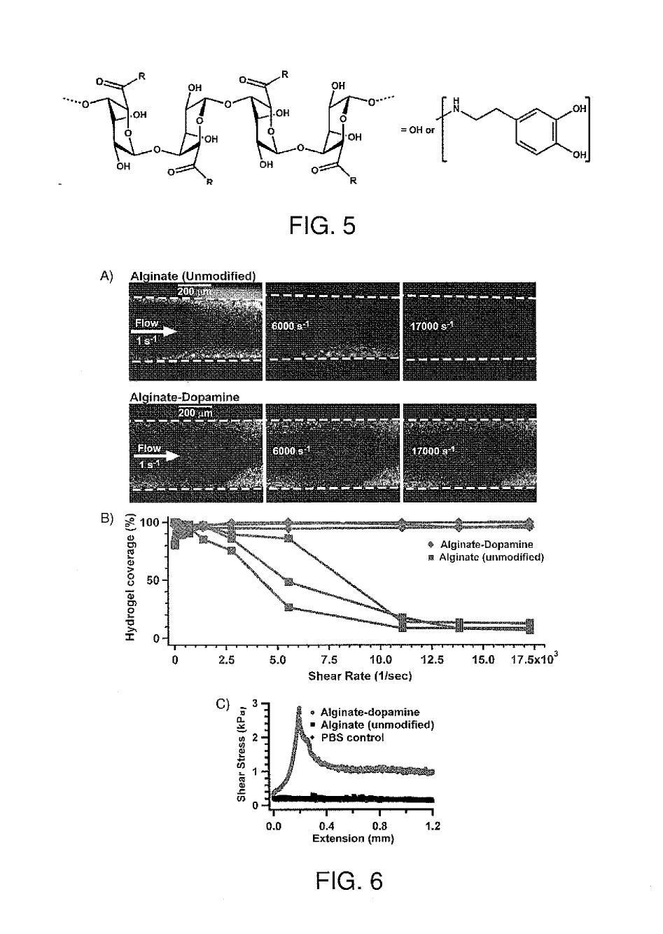

The following example provides information regarding the synthesis of alginate-dopamine materials. FIG. 5 shows the structure of alginate-dopamine. Alginate (400 mg, Protantol LF 10/60, FMC Biopolymer) was purified prior to the reaction by dialysis using spectra/por membrane tubing (MWCO 6-8 k, Spectrum Laboratories, Inc.). The purified alginate was dissolved in 80 mL of purified water. N-(3-dimethylaminopropyl)-M-ethylcarbodiimide hydrochloride (EDC) (437.1 mg, Sigma-Aldrich, Inc.) and N-hydroxysulfosuccinimide sodium salt (Sulfo-NHS) (495.1 mg, Sigma-Aldrich, Inc.) were each dissolved in 2 mL of purified water and added to the alginate solution. 3-Hydroxytyramine hydrochloride (Dopamine) (432.4 mg, Sigma-Aldrich, Inc.) was dissolved in 2 mL of purified water and added to the alginate reaction mixture. The pH of the mixture was adjusted to 5.5-6 using a solution of NaOH. The reaction mixture was stirred overnight at room temperature to produce alginate dopamine. Purification of alginate-dopamine was accomplished first by dialysis (spectra/por membrane tubing, MWCO 6-8 k, Spectrum Laboratories, Inc.) three times for a total of ˜24 h of dialysis. The solution was then concentrated using centrifugal filters (CentriPrep MWCO 10,000 kDa, Millipore, Inc.) by centrifuging at 800×g until the volume was reduced to 25 mL (−2 h). Alginate-dopamine was further purified using centrifugal desalting columns (PD-10, GE healthcare) using the vendor's procedure. Water was removed from the sample by freeze-drying. The yield was 189 mg of a white fluffy solid. Incorporation of dopamine into alginate was confirmed by H¹ NMR, with ˜30% of the saccharide units modified with dopamine.

Alginate-dopamine has the following characteristics which make it ideal for a vascular paint: i) unmodified alginate is biocompatible and used clinically, ii) gelation can be controlled by two distinct mechanisms: crosslinking via the dopamine moieties and via ionic interactions using divalent cations, iii) a high dopamine content can be achieved in the gel compared to previously prepared PEG-DOPA hydrogels.

### Example 3

The following example provides information regarding the synthesis of hyaluronic acid-dopamine (HA-dopamine). To prepare HA-dopamine, the procedure described in Example 2 was used, replacing alginate with hyaluronic acid (HA). The following quantities were used: HA (50 mg, EMB Biosciences, Inc.), EDC (109.3 mg), Sulfo-NHS (123.8 mg), and dopamine (108.1 mg). The yield was 29 mg of a white fluffy solid.

### Example 4

The following example provides information regarding the gelation of adhesive hydrogels blended with fluorescent nanoparticles. Alginate-dopamine or HA-dopamine was dissolved in phosphate buffered saline (PBS) (5% weight/volume for alginate dopamine, or 2.5% for HA-dopamine). A gelation solution was prepared to both induce crosslinking of the alginate-dopamine or HA-dopamine and incorporate fluorescent nanoparticles into the gel. The gelation solution was prepared by combining 100 of a solution of sodium periodate (NaIO₄, 19.125 mg/mL in PBS, Sigma-Aldrich, Inc.), 20 μL of a solution of suspended fluorescent nanoparticles (200 nm diameter, emission at 680 nm, solution of 2% solids, Invitrogen, Inc.), and a solution of NaOH (0.4 M, 12 μL added for alginate-dopamine or 1 μL added for HA-dopamine). The alginate-dopamine or HA-dopamine solution and gelation solution were mixed 3:1 to induce gelation in 10-20 minutes. The gelation time could be varied by adjusting the amount of NaOH that was added. Using pH paper, the pH of the alginate-dopamine pre-gel was ˜8.5.

### Example 5

The following example provides information regarding gelation in situ in the artery of a living mouse. A small incision was made into a surgically-exposed and ligated artery of a black C57 mouse. (JAX strain # 000664, Jackson Laboratories, Inc.) or an ApoE^{-/-} mouse (JAX strain # 002052, Jackson Laboratories, Inc.). A catheter composed of modified polytetrafluoroethylene (PTFE) tubing (inner diameter 150 μm, outer diameter 300 μm, Zeus, Inc.) was inserted into the artery and moved through the artery to the desired location. The hydrogel (e.g., alginate-dopamine, HA-dopamine) solution were mixed with the gelation solution and injected through the catheter. This pre-gel accumulated between the catheter and the vessel wall. The solution was allowed to gel, and the catheter was removed 10 min after gelation. The presence of the gel was confirmed using intravital microscopy using an Olympus OV-110 instrument.

### Example 6

The following example provides information in vitro experiments using alginate-dopamine material. Microfluidic devices were used to demonstrate the ability of the alginate-dopamine hydrogel to adhere to endothelial cells under physiological flow rates. Devices were prepared by growing human umbilical vein endothelial cells (HUVEC) inside of the microfluidic channels (FIGS. 2A-2B). FIG. 2C shows a schematic of a microfluidic device used to compare the adhesion of alginate and alginate-dopamine. The cells were coated with the alginate-dopamine hydrogel containing both green and red fluorescent particles by injecting the gel through a catheter. Growth media was then flowed through the devices at various flow and shear rates and the amount of fluorescence in the channel was measured to determine at which shear rate the adhesion of the gel fails (FIGS. 6A-6B). Specifically, FIG. 6A shows the timelapse fluorescent images of hydrogels on HUVEC cells with increasing shear rate. The nuclei of the cells were stained blue (shown as larger, darker grey spots), while the hydrogel contained both green fluorescent particles (1 um (micrometer) diameter; shown as smaller, light grey/white spots) and near-IR (magenta) fluorescent particles (0.2 um diameter; shown as smaller, light grey/white spots). The top panel shows the unmodified (control) alginate hydrogel break from the gel prior to a shear rate of 6000 s⁻¹, while the bottom panel shows the alginate-dopamine hydrogel adhered in the device at shear rates up to 17000 s⁻¹. FIG. 6B shows a graph quantifying the fluorescence intensity of the gels (% hydrogel coverage) at increasing shear rates. FIG. 6C shows a graph quantifying the adhesion of the hydrogels to glass slides in lap-shear by tension loading (a non-microfluidic method). As a control experiment the adhesion of unmodified alginate crosslinked was tested. This unmodified alginate broke off the HUVEC at a shear rate of about 1000 s⁻¹, which is a shear rate at the upper limit of physiological shear rates. In contrast, alginate-dopamine hydrogels were never observed to break off of the cells, even at shear rates as high as about 17000 s⁻¹. The alginate-dopamine gel was highly stable under flow—the gel remained completely adhered even when exposed to flowing solution for greater than about 12 hrs (10 s⁻¹) followed by bursts of shear rates up to about 17000 s⁻¹.

### Example 7

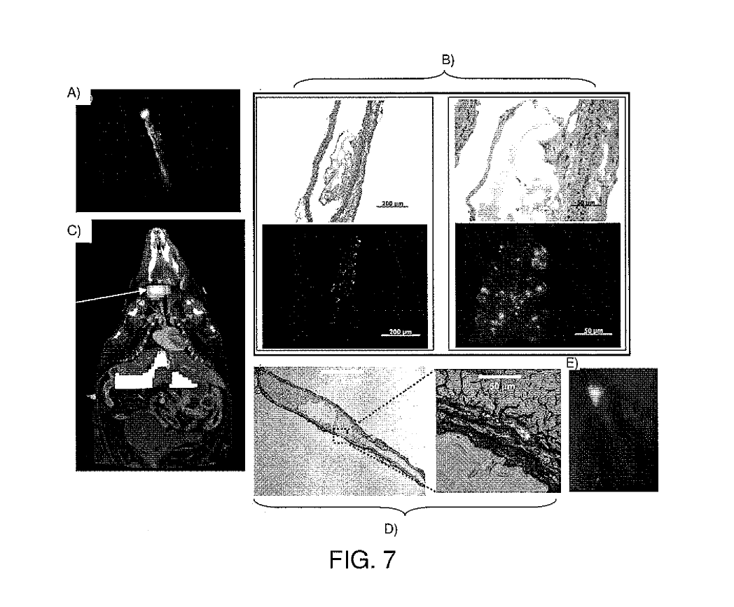

The following example provides information regarding the coating of alginate-dopamine hydrogel on the inside the carotid arteries of mice. The alginate-dopamine hydrogel adheres very strongly to the carotid arteries of mice over time. This was demonstrated using three methods to monitor the fluorescence of the gel. Intravital microscopy showed that the hydrogel was stable inside the carotid artery of a live mouse for 10-30 days (FIG. 7A). Specifically, FIG. 7A shows intravital microscopy image of the carotid artery of a live mouse 10 days after coating with the alginate-dopamine hydrogel. Histological analysis also confirmed that the hydrogel remained adhered to the vessel wall for over 30 days. FIG. 7B shows H & E histological sections (top) and fluorescence images (bottom) of a carotid artery coated with the hydrogel (shown as white in the fluorescence images) for 34 days. In a separate experiment, fluorescence-meditated tomography with computed tomography (FMT-CT) also confirmed the presence of the hydrogel in the carotid artery of a living mouse. FIG. 7C shows FMT-CT image of the hydrogel inside the carotid. The arrow indicates the area of fluorescence. Histological analysis of excised carotid arteries from separate mice show that the thickness of the gel can be as thin as 5-30 μm (FIG. 7D). FIG. 7D shows histological sections (length-wise section) showing a 5-30 um coating of the hydrogel (white dots) along the vessel wall. The hydrogel can also be locally coated on atherosclerotic plaques in a mouse model of atherosclerosis (ApoE knockout) (FIG. 7E). FIG. 7E shows intravital microscopy of hydrogel (white) coated on an atherosclerotic plaque (grey indicates background fluorescence from carotid artery and surrounding tissue).

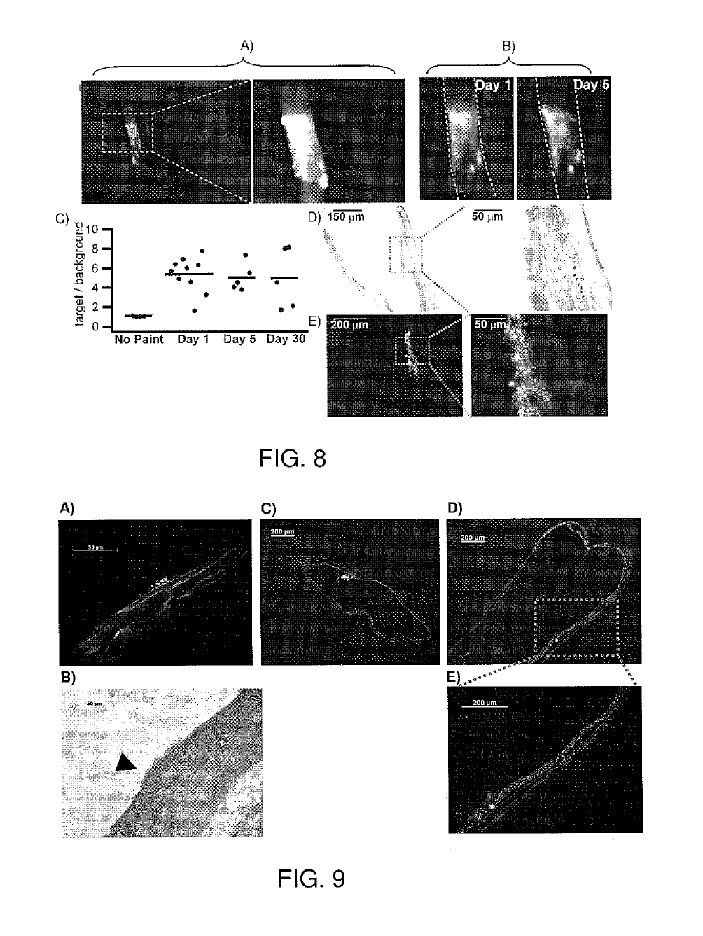

As another example, hydrogel may be painted in the carotid allies of living mice and can remain permanently adhered. FIG. 8A shows fluorescence images of a carotid artery in a living mouse painted with alginate-dopamine (white). A fluorescent dye (light grey) was injected intravenously 15 sec prior to imaging to monitor blood flow through the vessel. FIG. 8B shows fluorescence images of alginate-dopamine painted in the carotid artery at day 1 and day 5 after deposition. FIG. 8C shows a chart quantifying the amount of hydrogel in vessels over time. FIG. 8D shows histological sections (H&E staining) of a carotid artery painted for 28 days. FIG. 8E shows fluorescence images from histological section adjacent to those of FIG. 8D.

As yet another example, various lengths of blood vessels were painted with alginate-dopamine. FIG. 9 shows overlaid fluorescence images of histological section of alginate-dopamine containing fluorescent nanoparticles (white spots) and autofluorescence of the blood vessel wall (grey). FIG. 9A shows a coating of alginate-dopamine on the 10 um scale. FIG. 9B shows histological (H&E stain) section for sample in FIG. 9A. The arrow indicates the location of the hydrogel. FIG. 9C shows a coating of alginate-dopamine on the 100 um scale. FIGS. 9D-9E show coatings of alginate-dopamine on the 1 mm scale.

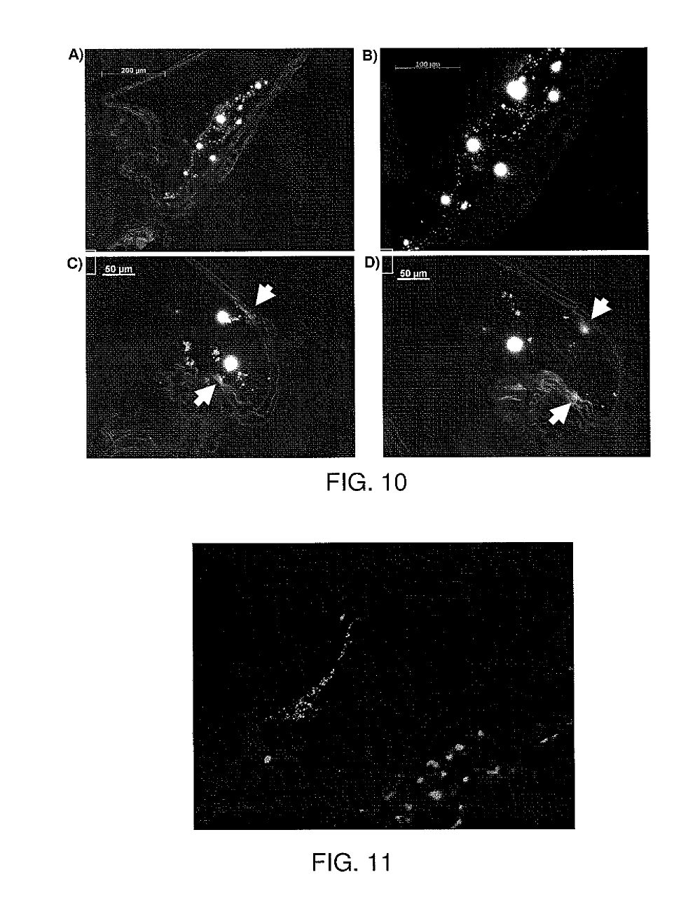

As still yet another example, FIG. 10 shows painting bifurcation regions of blood vessels with degradable particles. Overlaid fluorescence images of histological sections are shown of the alginate-dopamine hydrogel containing fluorescent nanoparticles (smaller grey/white spots), poly(lactic-co-glycolic acid) microparticles containing a fluorescent dye, BODIPY, (large white spots) and autofluorescence of the blood vessel wall (grey background). The hydrogel was coated in the internal carotid artery at the bifurcation of the common carotid artery into the internal and external carotid arteries. Specifically, FIG. 10A shows a lower magnification image of a painted bifurcation. FIG. 10B shows a higher magnification image of the section from FIG. 10A. FIG. 10C shows an image of the blood vessel wall stained with BODIPY that was release from nearby particles. FIG. 10D shows an image of a section adjacent to FIG. 10C confirming that the vessel wall was stained with BODIPY.

FIG. 11 gives images relating to painting atherosclerotic plaques. Overlaid fluorescence images of histological sections of alginate-dopamine containing fluorescent nanoparticles (smaller grey/white spots) and background fluorescence of a carotid artery (larger grey/white spots). The mouse was an ApoE−/− mutant fed a high cholesterol diet for over one year to induce atherosclerotic plaque formation.

### Example 8

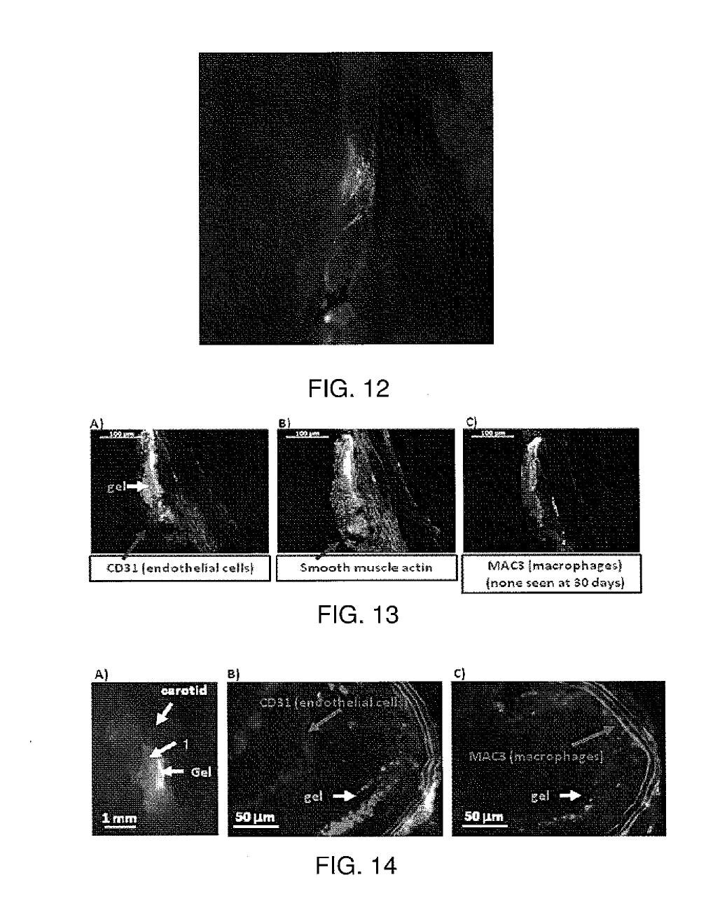

The following example provides information in vivo experiments using HA-dopamine material. FIG. 12 shows an image of a carotid artery painted with hyaluronic acid-dopamine. Overlaid fluorescence images of a live mouse are shown. Images show fluorescent nanoparticles incorporated into the hyaluronic acid-dopamine hydrogel (white spots), and of background fluorescence of the blood vessels of the mouse (grey background).

### Example 9

As shown in FIG. 13, alginate-dopamine (administered using methods similar to as described in Example 5) adhered strongly to carotid arteries of wild type mice for over four weeks and the blood vessel remains viable. Fluorescence images from immunohistological staining of sectioned vessels are shown. The small dots indicates specific staining for the indicated proteins and cells. Whiter areas indicate the location of the adhesive hydrogel. At 30 days after treatment, the vessel has grown endothelial cells over the gel (A), smooth muscle cells have grown around the gel (B), and no macrophages are seen around the gel (C). This indicates that the gel does not elicit a severe inflammatory response or necrosis in the blood vessel, in this embodiment.

As shown in FIG. 14, alginate-dopamine (administered using methods similar to as described in Example 5) was coated on atherosclerotic plaques. In FIG. 14A, fluorescence image of a carotid artery in a living mouse (an atherosclerosis model, mouse strain ApoE −/−) taken using intravital microscopy is shown. The image shows that the adhesive hydrogel (indicated by arrow) can be coated on a plaque (indicated by arrow 1, from a fluorescent substrate for cathepsin, a protease in plaques). FIGS. 14B-14C show fluorescence images from immunohistological staining of sectioned vessels. Fluorescence indicates specific staining for endothelial cells (B) or macrophages in the plaque (C) are indicated by arrow. Lighter colors indicated by arrows labeled gel show the regions where the hydrogen was present. The images show that the hydrogel can be coated on a macrophage rich plaque and after 24 days the vessels grows a new layer of endothelial cells over the gel and plaque.

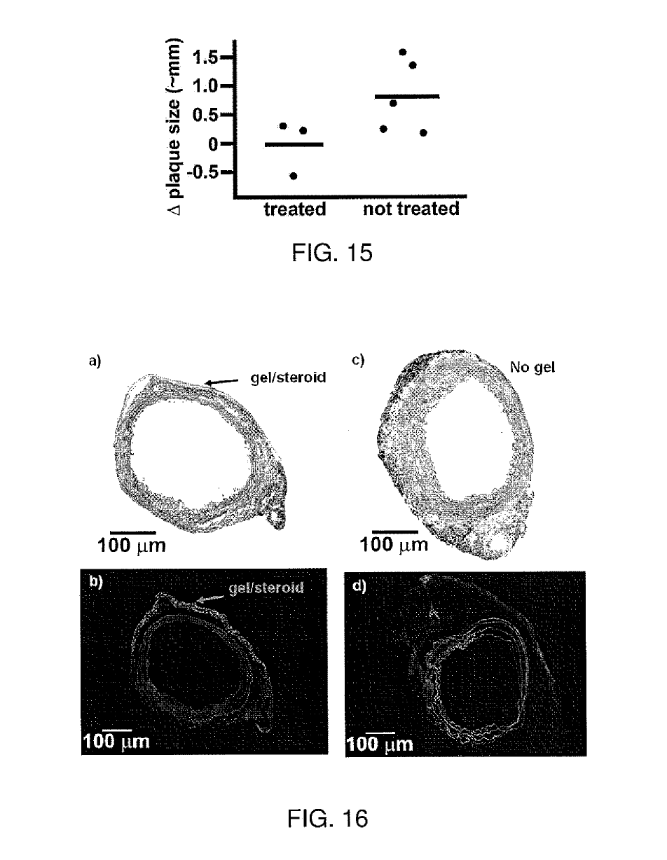

FIG. 15 gives information regarding atherosclerotic plaques growth in mice. The atherosclerotic plaques grow less in mice vessels treated with the adhesive hydrogel containing dexamethasone. Mouse vessels were harvested 3-4 weeks after treatment and the plaque size was measured from images taken using intravital microscopy.

### Example 10

The compositions described herein may be used to form coatings (e.g., uniform coatings) on the outside of blood vessels. Coating of the outside of blood vessels was accomplished by surgically dissecting the tissue and exposing the arteries of interest. The adhesive liquid pre-hydrogel was then pipetted onto the artery and allowed to completely gel, then the incision was closed. This procedure was performed with the alginate-dopamine hydrogel, both with and without a steroid, dexamethasone, loaded in the gel. FIG. 16 shows images of the coating the outside of blood vessels with adhesive hydrogels. FIGS. 16A-16B show a carotid artery of a mouse coated with alginate-dopamine containing a steroid. In FIG. 16B, the gel is visible as the small white dots. FIGS. 16C-16D show control sample of a carotid artery that was not coated with adhesive hydrogel or steroid.

Coating the outside of vessels can be used to treat any of the diseases previously described, such as atherosclerosis and aneurisms. In addition, the coating the outside of vessels may also be useful for treating other damaged portions of vessels. The hydrogel can be used to treat regions where blood vessels are surgically connected together (anastomoses) or where vessels are connected to artificial grafts. It can also be used to treat areas where blood vessels have been punctured. The adhesive hydrogel can be loaded with various therapeutics, such as those described in preceding claims, in order to help the vessel heal.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. A composition, comprising:
a polysaccharide comprising a plurality of functional groups having the formula: 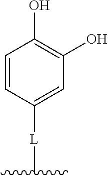
wherein L is a linker associating the functional group to the polysaccharide backbone.

2. A method, comprising:
providing a composition comprising a polymer and a plurality of functional groups having the formula: 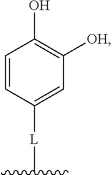
wherein L is a linker associating the functional groups to the polymer; and
coating at least a portion of an interior surface of a hollow organ or tissue lumen with the composition.

3. (canceled)

4. A method, comprising:
providing a composition comprising a polymer and a plurality of functional groups having the formula: 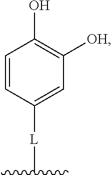
wherein L is a linker associating the functional groups to the polymer; and
coating at least a portion of a body surface with the composition.

5. (canceled)

6. (canceled)

7. The composition of claim 1, wherein the polysaccharide alginate.

8. The composition of claim 1, wherein the functional groups have the formula: 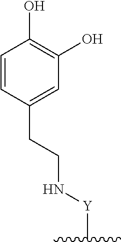
wherein Y is a linker linking the functional group to the polysaccharide backbone, or optionally absent.

9. The composition of claim 1, wherein the functional groups have the formula: 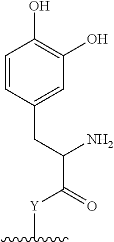
wherein Y is a linker linking the functional group to the polysaccharide backbone, or optionally absent.

10. The of claim 2, wherein the polymer comprises hyaluronic acid.

11. The method of claim 2, wherein the polymer comprises polyethylene glycol.

12. The method of claim 2, wherein the polymer is a polysaccharide.

13. The method of claim 2, wherein the composition has the structure: 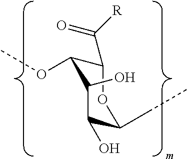
wherein, each R is OH or a group comprising dopamine or a 3,4-dihydroxyphenylalanine and m is an integer between 1 and about 10,000.

14. The method of claim 13, wherein each R is OH or a group having the structure:

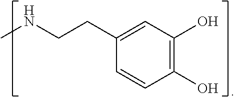

15. The composition of claim 1, wherein the composition has the structure: 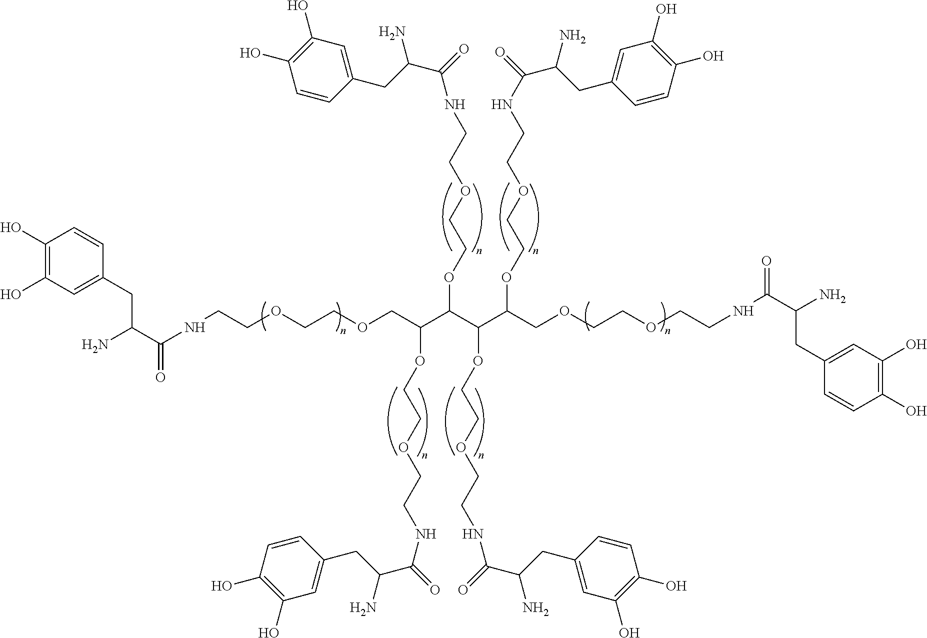
wherein each n is the same or different and is an integer between 1 and 100.

16. The composition of claim 15, wherein n is an integer between about 1 and about 50.

17. The composition of claim 15, wherein n is an integer between about 10 and about 100.

18. The method of claim 2, wherein the composition is provided as a solution.

19. The method of claim 16, wherein the solution is exposed to a crosslinking agent during the providing step.

20. The method of claim 19, wherein the polymer is crosslinked by the crosslinking agent.

21. The method of claim 20, wherein the composition is at least partially solidified following the providing step.

22. The method of claim 21, wherein the composition is at least partially solidified by the formation of a plurality of crosslinks between polymer chains.

23. The method of claim 21, wherein the partially solidified composition is a gel.

24-43. (canceled)
